(19) United States
(12) Patent Application Publication
(10) Pub. No.: US 2007/0244545 A1
(43) Pub. Date: Oct. 18, 2007

(54) Prosthetic Conduit With Radiopaque Symmetry Indicators

(75) Inventors: Matthew J. Birdsall, Santa Rosa, CA (US); Mark J. Dolan, Santa Rosa, CA (US); Darrel Untereker, Oak Grove, MN (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/279,776

(22) Filed: Apr. 14, 2006

(51) Int. Cl. A61F 2/24

(52) U.S. Cl. 623/1.26; 623/1.34

(57) ABSTRACT

A system and method for treating a vascular condition includes a conduit having an elongate tubular member with an outer surface and an inner surface, the inner surface defines a conduit lumen. The system further includes at least one symmetry indicator attached to the elongate tubular member and a replacement valve device. The replacement valve device includes a prosthetic valve connected to an expandable support structure. The replacement valve device is positioned within the conduit lumen adjacent the inner surface.

### TECHNICAL FIELD

This invention relates generally to medical devices for treating cardiac valve abnormalities, and particularly to a pulmonary valve replacement system and method of employing the same.

### BACKGROUND OF THE INVENTION

Heart valves, such as the mitral, tricuspid, aortic and pulmonary valves, are sometimes damaged by disease or by aging, resulting in problems with the proper functioning of the valve. Heart valve problems generally take one of two forms: stenosis, in which a valve does not open completely or the opening is too small, resulting in restricted blood flow; or insufficiency, in which blood leaks backward across a valve when it should be closed.

The pulmonary valve regulates blood flow between the right ventricle and the pulmonary artery, controlling blood flow between the heart and the lungs. Pulmonary valve stenosis is frequently due to a narrowing of the pulmonary valve or the pulmonary artery distal to the valve. This narrowing causes the right side of the heart to exert more pressure to provide sufficient flow to the lungs. Over time, the right ventricle enlarges, which leads to congestive heart failure (CHF). In severe cases, the CHF results in clinical symptoms including shortness of breath, fatigue, chest pain, fainting, heart murmur, and in babies, poor weight gain. Pulmonary valve stenosis most commonly results from a congenital defect, and is present at birth, but is also associated with rheumatic fever, endocarditis, and other conditions that cause damage to or scarring of the pulmonary valve. Valve replacement may be required in severe cases to restore cardiac function.

Previously, valve repair or replacement required open-heart surgery with its attendant risks, expense, and extended recovery time. Open-heart surgery also requires cardiopulmonary bypass with risk of thrombosis, stroke, and infarction. More recently, flexible valve prostheses and various delivery devices have been developed so that replacement valves can be implanted transvenously using minimally invasive techniques. As a consequence, replacement of the pulmonary valve has become a treatment option for pulmonary valve stenosis.

The most severe consequences of pulmonary valve stenosis occur in infants and young children when the condition results from a congenital defect. Frequently, the pulmonary valve must be replaced with a prosthetic valve when the child is young, usually less than five years of age. However, as the child grows, the valve can become too small to accommodate the blood flow to the lungs that is needed to meet the increasing energy demands of the growing child, and it may then need to be replaced with a larger valve. Alternatively, in a patient of any age, the implanted valve may fail to function properly due to calcium buildup and have to be replaced. In either case, repeated surgical or transvenous procedures are required.

To address the need for pulmonary valve replacement, various implantable pulmonary valve prostheses, delivery devices and surgical techniques have been developed and are presently in use. One such prosthesis is a bioprosthetic, valved conduit comprising a glutaraldehyde treated bovine jugular vein containing a natural, trileaflet venous valve, and sinus. A similar device is composed of a porcine aortic valve sutured into the center of a woven fabric conduit. A common conduit used in valve replacement procedures is a homograft, which is a vessel harvested from a cadaver. Valve replacement using either of these devices requires thoracotomy and cardiopulmonary bypass.

When the valve in the prostheses must be replaced, for the reasons described above or other reasons, an additional surgery is required. Because many patients undergo their first procedure at a very young age, they often undergo numerous procedures by the time they reach adulthood. These surgical replacement procedures are physically and emotionally taxing, and a number of patients choose to forgo further procedures after they are old enough to make their own medical decisions.

Recently, implantable stented valves have been developed that can be delivered transvenously using a catheter-based delivery system. These stented valves comprise a collapsible valve attached to the interior of a tubular frame or stent. The valve can be any of the valve prostheses described above, or it can be any other suitable valve. In the case of valves in harvested vessels, the vessel can be of sufficient length to extend beyond both sides of the valve such that it extends to both ends of the valve support stent.

The stented valves can also comprise a tubular portion or “stent graft” that can be attached to the interior or exterior of the stent to provide a generally tubular internal passage for the flow of blood when the leaflets are open. The graft can be separate from the valve and it can be made from any suitable biocompatible material including, but not limited to, fabric, a homograft, porcine vessels, bovine vessels, and equine vessels.

The stent portion of the device can be reduced in diameter, mounted on a catheter, and advanced through the circulatory system of the patient. The stent portion can be either self-expanding or balloon expandable. In either case, the stented valve can be positioned at the delivery site, where the stent portion is expanded against the wall of a previously implanted prostheses or a native vessel to hold the valve firmly in place.

One embodiment of a stented valve is disclosed in U.S. Pat. No. 5,957,949 titled “Percutaneous Placement Valve Stent” to Leonhardt, et al, the contents of which are incorporated herein by reference.

One obstacle for implanting a stented valve within a conduit is that, over time, the conduit may become misshapen or asymmetrical. While this asymmetry is not necessarily damaging to the patient it is, however, problematic for delivering and positioning stented correctly within the conduit. Another obstacle is that, prior to placement of a stented valve it is difficult for a clinician to determine whether the conduit is misshapen and the extent of any deformation that may exist.

It would be desirable, therefore, to provide an implantable pulmonary valve that would overcome the limitations and disadvantages in the devices described above.

### SUMMARY OF THE INVENTION

It is an object of the present invention to provide a heart valve replacement system having at least a conduit and a replacement valve device. The conduit includes a conduit symmetry indicator. The replacement valve device includes a prosthetic valve attached to a support structure.

The system and the prosthetic valve will be described herein as being used for replacing a pulmonary valve. The pulmonary valve is also known to those having skill in the art as the “pulmonic valve” and as used herein, those terms shall be considered to mean the same thing.

Thus, one aspect of the present invention provides a pulmonary valve replacement system. The pulmonary valve replacement system includes a conduit comprising an elongate tubular member having an outer surface and an inner surface, the inner surface defines a conduit lumen. The system further includes at least one symmetry indicator attached to the elongate tubular member and a replacement valve device. The replacement valve device includes a prosthetic valve connected to an expandable support structure. The replacement valve device is positioned within the conduit lumen adjacent the inner surface.

Another aspect of the invention provides a prosthetic conduit device for treating a vascular condition. The device includes a conduit comprising an elongate tubular member having an outer surface and an inner surface, the inner surface defining a conduit lumen and at least one symmetry indicator attached to the elongate tubular member.

Another aspect of the invention provides a method for treating a vascular condition. The method comprises inserting a conduit having a radiopaque conduit symmetry device into a target region of a vessel, visualizing the radiopaque conduit symmetry device and determining conduit symmetry based on the visualization of the radiopaque conduit symmetry device. The method further includes delivering a stented valve into the conduit lumen, the stented valve includes a prosthetic valve connected to an expandable support structure and expanding the stented valve into contact with the inner wall of the conduit.

The present invention is illustrated by the accompanying drawings of various embodiments and the detailed description given below. The drawings should not be taken to limit the invention to the specific embodiments, but are for explanation and understanding. The detailed description and drawings are merely illustrative of the invention rather than limiting, the scope of the invention being defined by the appended claims and equivalents thereof The drawings are not to scale. The foregoing aspects and other attendant advantages of the present invention will become more readily appreciated by the detailed description taken in conjunction with the accompanying drawings.

### BRIEF DESCRIPTION OF THE DRAWINGS

FIG. 1 is a schematic interior view of a human heart showing the functioning of the four heart valves;

FIG. 2A is a schematic view showing the placement of a pulmonary conduit, as is known in the prior art;

FIG. 2B is a schematic view showing attachment of a pulmonary conduit to the pulmonary artery, as is known in the prior art;

FIG. 2C is a schematic view showing attachment of a pulmonary conduit to the heart, as is known in the prior art;

FIG. 3 is a schematic view of one embodiment of a prosthetic valve device situated in a conduit, in accordance with the present invention;

FIG. 4 is a schematic view of one embodiment of a prosthetic valve device having a conduit symmetry indicator, in accordance with the present invention;

FIGS. 5A and 5B are schematic views showing a detailed portion of the conduit symmetry indicator illustrated in FIG. 4;

FIGS. 6A to 6C are schematic views of a prosthetic valve device having another embodiment of a conduit symmetry indicator, in accordance with the present invention;

FIGS. 7A to 7B are schematic views of a prosthetic valve device having another embodiment of a conduit symmetry indicator, in accordance with the present invention; and

FIG. 8 is a flow diagram of one embodiment of a method of treating a vascular condition in accordance with the present invention.

### DESCRIPTION OF THE PRESENTLY PREFERRED EMBODIMENTS

The invention will now be described by reference to the drawings wherein like numbers refer to like structures.

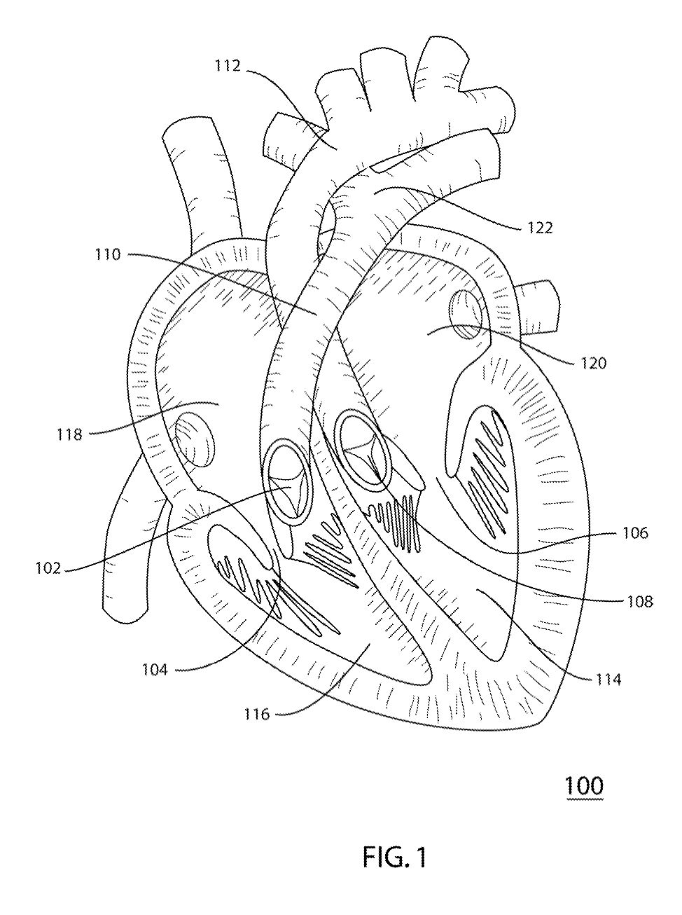

Referring to the drawings, FIG. 1 is a schematic representation of the interior of human heart 100. Human heart 100 includes four valves that work in synchrony to control the flow of blood through the heart. Tricuspid valve 104, situated between right atrium 118 and right ventricle 116, and mitral valve 106, between left atrium 120 and left ventricle 114 facilitate filling of ventricles 116 and 114 on the right and left sides, respectively, of heart 100. Aortic valve 108 is situated at the junction between aorta 112 and left ventricle 114 and facilitates blood flow from heart 100, through aorta 112 to the peripheral circulation.

Pulmonary valve 102 is situated at the junction of right ventricle 116 and pulmonary artery 110 and facilitates blood flow from heart 100 through the pulmonary artery 110 to the lungs for oxygenation. The four valves work by opening and closing in harmony with each other. During diastole, tricuspid valve 104 and mitral valve 106 open and allow blood flow into ventricles 114 and 116, and the pulmonic valve and aortic valve are closed. During systole, shown in FIG. 1, aortic valve 108 and pulmonary valve 102 open and allow blood flow from left ventricle 114, and right ventricle 116 into aorta 112 and pulmonary 110, respectively.

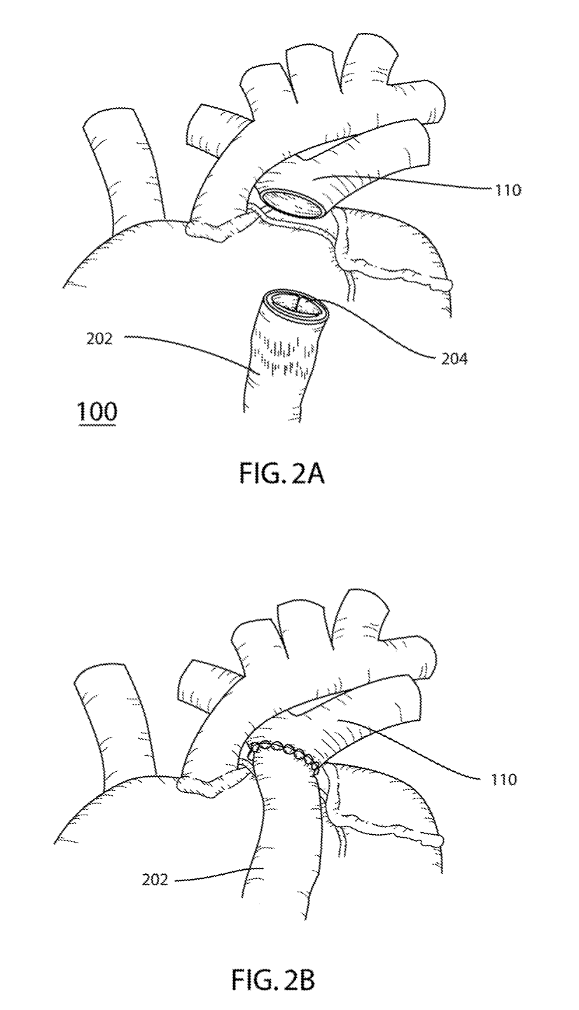

The right ventricular outflow tract is the segment of pulmonary artery 110 that includes pulmonary valve 102 and extends to branch point 122, where pulmonary artery 110 forms left and right branches that carry blood to the left and right lungs respectively. A defective pulmonary valve or other abnormalities of the pulmonary artery that impede blood flow from the heart to the lungs sometimes require surgical repair or replacement of the right ventricular outflow tract with prosthetic conduit 202, as shown in FIG. 2A-C.

Such conduits comprise tubular structures of biocompatible materials, with a hemocompatible interior surface. Examples of appropriate biocompatible materials include polytetrafluoroethylene (PTFE), woven polyester fibers such as Dacron® fibers (E. I. Du Pont De Nemours & Co., Inc.), and bovine vein crosslinked with glutaraldehyde. One common conduit is a homograft, which is a vessel harvested from a cadaver and treated for implantation into a recipient's body. These conduits may contain a valve at a fixed position within the interior lumen of the conduit that functions as a replacement pulmonary valve. One such conduit 202 comprises a bovine jugular vein with a trileaflet venous valve preserved in buffered glutaraldehyde. Other valves are made of xeno-pericardial tissue and are attached to the wall of the lumen of the conduit. Still other valves may be made at least partially from some synthetic material.

As shown in FIGS. 2A and 2B, conduit 202, which houses valve 204 within its inner lumen, is installed within a patient by sewing the distal end of conduit 202 to pulmonary artery 110, and, as shown in FIG. 2C, attaching the proximal end of conduit 202 to heart 100 so that the lumen of conduit 202 connects to right ventricle 1 16.

Over time, implanted prosthetic conduits and valves are frequently subject to calcification, causing the affected conduit or valve to lose flexibility, become misshapen, and lose the ability to function effectively. Additional problems are encountered when prosthetic valves are implanted in young children. As the child grows, the valve will ultimately be too small to handle the increased volume of blood flowing from the heart to the lungs. In either case, the valve needs to be replaced.

The current invention discloses devices and methods for percutaneous catheter based placement of stented valves for regulating blood flow through a pulmonary artery. In a preferred embodiment, the valves are attached to an expandable support structure and they are placed in a valved conduit that is been attached to the pulmonary artery, and that is in fluid communication with the right ventricle of a heart. The support structure can be expanded such that any pre-existing valve in the conduit is not disturbed, or it can be expanded such that any pre-existing valve is pinned between the support structure and the interior wall of the conduit.

The delivery catheter carrying the stented valve is passed through the venous system and into a patient's right ventricle. This may be accomplished by inserting the delivery catheter into either the jugular vein or the subclavian vein and passing it through superior vena cava into right atrium. The catheter is then passed through the tricuspid valve, into right ventricle, and out of the ventricle into the conduit. Alternatively, the catheter may be inserted into the femoral vein and passed through the common iliac vein and the inferior vena cava into the right atrium, then through the tricuspid valve, into the right ventricle and out into the conduit. The catheters used for the procedures described herein may include radiopaque markers as are known in the art, and the procedure may be visualized using fluoroscopy, echocardiography, ultrasound, or other suitable means of visualization.

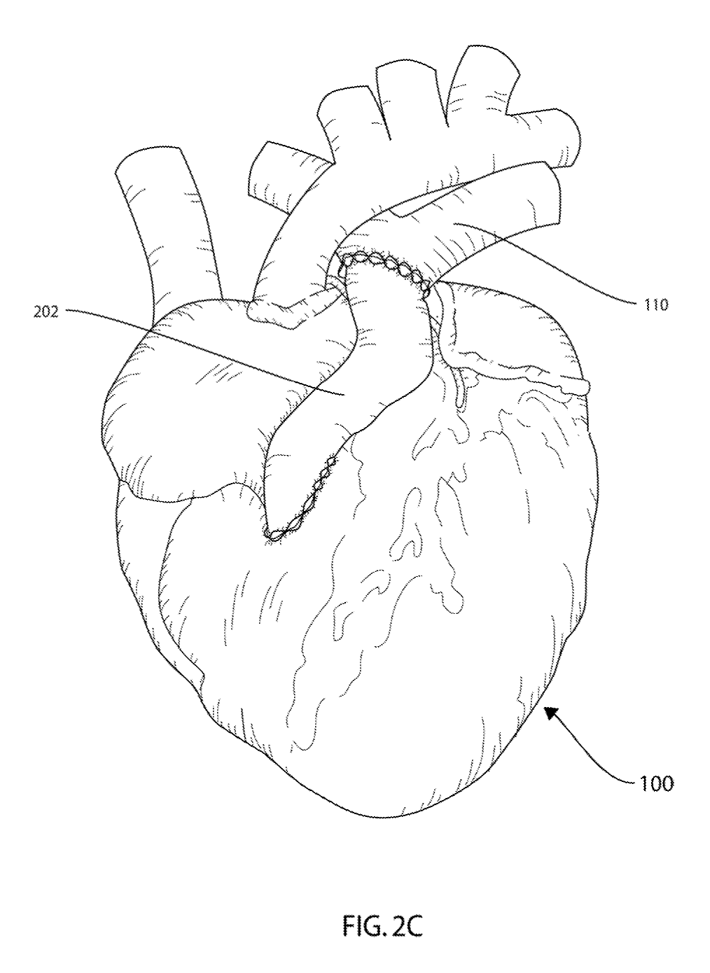

FIG. 3 illustrates a cross section of one embodiment of a system 300 for treating a vascular condition within heart 100 illustrated in FIG. 1. System 300 illustrated in FIG. 3 is described herein with reference to a bioprosthetic conduit for replacing a portion of a pulmonary artery. Those with skill in the art will recognize that the invention may be adapted to other vessels of a body that require a replacement valve.

System 300 includes a conduit 310 and a stented valve 320. Stented valve 320 comprises a support structure 322 and a prosthetic valve 324 operably connected to support structure 322.

Conduit 310 comprises an elongate tubular structure that includes an inner wall 312 that defines a lumen 314. Lumen 314 allows fluid communication between the right ventricle and the pulmonary artery. Conduit 310 includes a first end 316 for attaching to ventricle 110 and a second end 318 for attaching to pulmonary artery 122.

In one embodiment of the invention, support structure 322 is an expandable stent made of a flexible, biocompatible material. The support structure 322 may be composed of self-expanding material and manufactured from, for example, a nickel titanium alloy and/or other alloy(s) that exhibit superelastic behavior. Other suitable materials for support structure 322 include, but are not limited to, a nitinol alloy, a stainless steel, and a cobalt-based alloy, such as an MP35N® alloy. Furthermore, the support structure 322 material may include polymeric biocompatible materials recognized in the art for such devices. Support structure 322 retains the stented valve 320 within the vascular conduit 302.

In one embodiment, prosthetic valve 324 comprises a bovine jugular vein with a trileaflet venous valve preserved in buffered glutaraldehyde. In other embodiments, prosthetic valve 324 comprises a valve made of synthetic materials and attached to support structure 322.

Stented valve 320 is compressed and disposed on an inflatable member 330, which is operably attached to a catheter 340. Catheter 340 delivers stented valve 320 endovascularly to a treatment site within the vascular conduit 302. Stented valve 320 is positioned within the vascular conduit 302 and then expanded with an inflatable member 330 into contact with the inner surface 304 of conduit 302.

In one embodiment, catheter 340 is an elongated tubular member manufactured from one or more polymeric materials, sometimes in combination with metallic reinforcement. In some applications (such as smaller, more tortuous arteries), it is desirable to construct the catheter from very flexible materials to facilitate advancement into intricate access locations. Numerous over-the-wire, rapid-exchange, and other catheter designs are known and may be adapted for use with the present invention. Catheter 340 can be secured at its proximal end to a suitable Luer fitting, and includes a distal rounded end 342 to reduce harmful contact with a vessel wall. Catheter 340 is manufactured from a material such as a thermoplastic elastomer, urethane, polymer, polypropylene, plastic, ethelene chlorotrifluoroethylene (ECTFE), polytetrafluoroethylene (PTFE), fluorinated ethylene propylene copolymer (FEP), nylon, Pebax® resin, Vestamid® nylon, Tecoflex® resin, Halar® resin, Hyflon® resin, Pellathane® resin, combinations thereof, and the like. Catheter 340 includes an aperture formed at the distal rounded end 342 allowing advancement over a guidewire 344.

In one embodiment, inflatable member 330 is any variety of balloon or other device capable of expanding stented valve 320. Inflatable member 330 is manufactured from any suitable material such as polyethylene, polyethylene terephthalate (PET), nylon, or the like. Those skilled in the art will recognize that the stented valve 320 may be expanded using a variety of means and that the present invention is not limited to balloon expansion.

Vascular conduit 302 is designed to be a long term implant and frequently can become calcified or subject to fibrotic ingrowth of tissue, either of which sometimes causes the vascular conduit 302 to become misshapen, so that its cross section is no longer round and symmetrical. Consequently, a stented valve 320 would not fit well within a misshapen and/or asymmetrical vascular conduit 302, and may be ineffective either because of blood flowing around the outside of stented valve 320, or because stented valve 320 cannot be aligned perpendicularly to the flow of blood through vascular conduit 302.

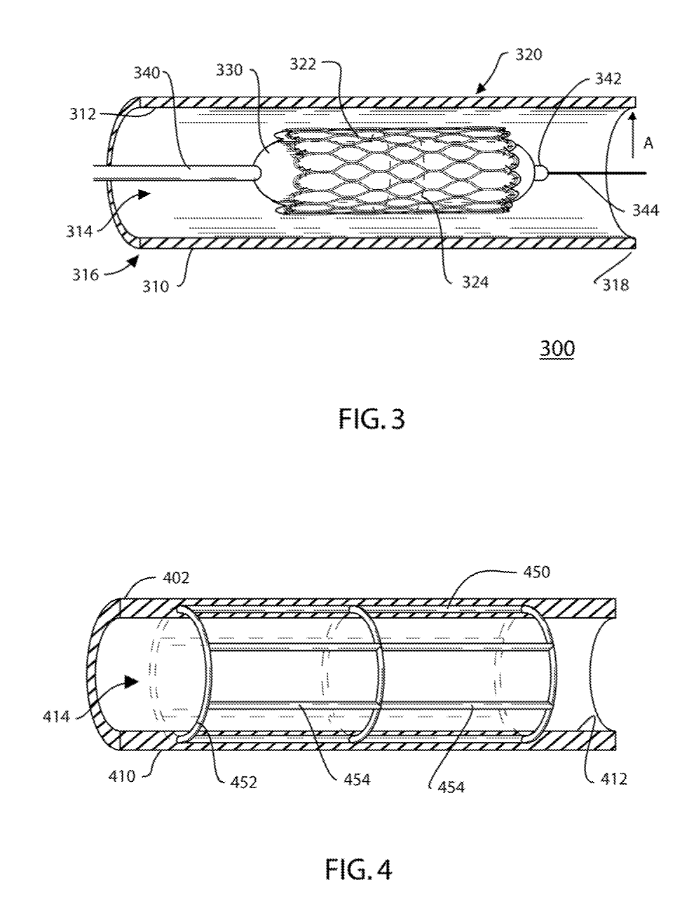

Referring to FIG. 4, illustrated is one embodiment of a vascular conduit 400 having a conduit symmetry indicator device 450. In one embodiment, vascular conduit 402 comprises an elongate tubular member having an outer surface 410 and an inner surface 412, the inner surface defining a conduit lumen 414. In one embodiment, conduit 402 is the same as or similar to conduit 202, described above.

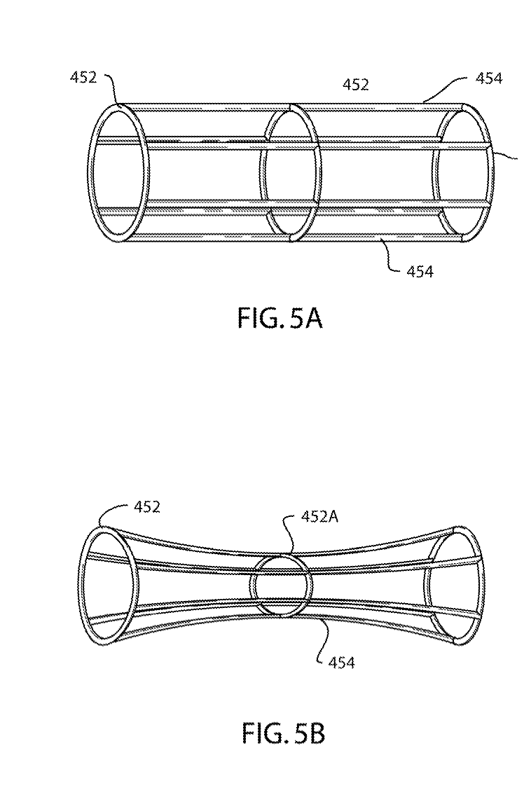

As illustrated in FIGS. 4 and 5A, conduit symmetry indicator device 450 comprises a plurality of radiopaque rings 452 connected by a plurality of radiopaque elongate members 454. Conduit symmetry indicator device 450 comprises metallic or polymeric radiopaque material having a high X-ray attenuation coefficient. Examples of suitable materials include, but are not limited to, barium sulfate and bismuth sub-carbonate for plastics. Suitable materials for metals include, but are not limited to, gold, platinum, and alloys thereof.

In one embodiment, rings 452 and elongate members 454 are disposed within the wall of vascular conduit 402. In one embodiment, rings 452 and elongate members 454 comprise filaments of radiopaque material woven into the material that comprises vascular conduit 402. The filaments may comprise an individual wire or a plurality of wires braided into a filament. The elongate members 454 are woven into the conduit material such that they are substantially parallel to the central axis of the conduit. The radiopaque filaments are woven into the material in such a manner as to provide a conduit symmetry indicator device 450 having a plurality of spaced apart rings 452 and a plurality of spaced apart elongate members 454 positioned around the circumference of the plurality of rings 452.

In another embodiment, rings 452 and elongate members 454 are threaded through the tissue comprising the vascular conduit 402 and secured to the conduit wall by, for example, sutures. For example, in a vascular conduit composed of bovine tissue, a filament of radiopaque material is threaded through and around the wall of the conduit to form a ring. This is repeated until the desired number of rings 452 are placed within the conduit wall. Next, a plurality of elongate members are threaded within the tissue of the conduit wall such that the elongate members are substantially parallel to the central axis of the conduit. In one embodiment, the elongate members 454 are secured to the plurality of rings 452, by for example, suturing.

FIG. 5A illustrates conduit symmetry indicator device 450 in a symmetrical non-misshapen state, as it would appear prior to implantation. FIG. 5B illustrates conduit symmetry indicator device 450 in an asymmetrical misshapen state. The distance between any two rings 452 or any two elongate members 454 may be set at a predetermined distance that is maintained in a symmetrical conduit. Based on this set distance, any deviation from that set distance determined during visualization of the conduit provides an indication that the vascular conduit is misshapen and/or asymmetrical. Additionally, the asymmetrical nature of an implanted conduit may be determined by visualization of the rings 452. A ring 452A (FIG. 5B) in a collapsed conduit will no longer be substantially circular but, instead, will be flattened to form a more oval shape. Visualization of an oval shape, then, determines that the conduit is no longer symmetrical and may need to be corrected before implantation of a stented valve. Conduit symmetry indicator device 450 may be visualized using fluoroscopy, echocardiography, ultrasound, or other suitable means of visualization.

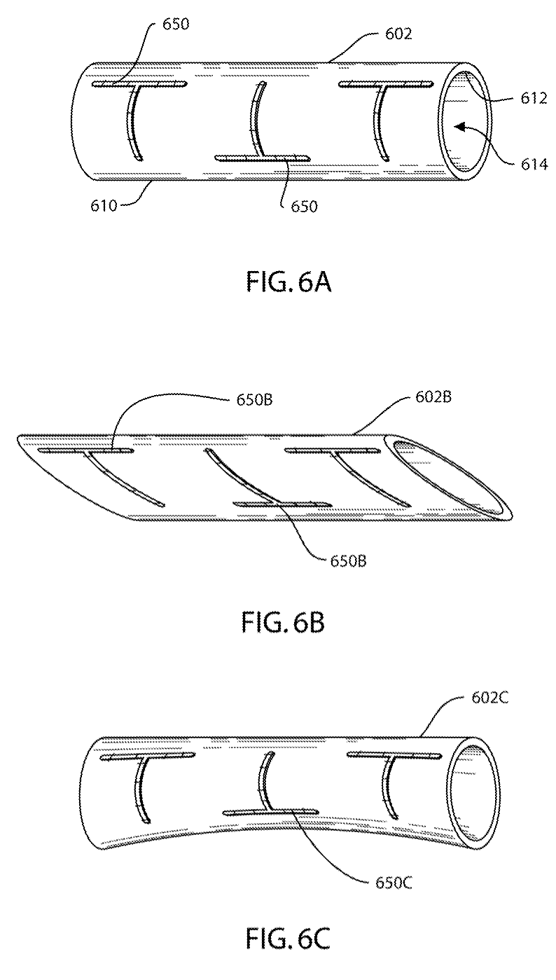

FIG. 6A illustrates another embodiment of a vascular conduit 602 having a plurality of conduit symmetry indicator devices 650. Vascular conduit 602 comprises an elongate tubular member having an outer surface 610 and an inner surface 612, the inner surface defining a conduit lumen 614. In one embodiment, conduit 602 is the same as or similar to conduit 202, described above.

Conduit symmetry indicator device 650 comprises a T-shaped radiopaque member attached to or embedded within the wall of vascular conduit 602. Conduit symmetry indicator device 650 comprises metallic or polymeric radiopaque material having a high X-ray attenuation coefficient. Examples of suitable materials include, but are not limited to, barium sulfate and bismuth sub-carbonate for plastics, and gold and platinum for metals. In one preferred embodiment conduit symmetry indicator device 650 comprises a filament of radiopaque material. The filament may be a wire or a plurality of wires braided into a filament. The filament is formed into a T-shaped configuration and attached to the vascular conduit 602. In another embodiment, conduit symmetry indicator device 650 comprises a plurality of radiopaque members attached to the vascular conduit in a T-shaped configuration. In an example, conduit symmetry indicator device 650 comprises a plurality of round radiopaque members attached to the outer surface of the vascular conduit in a T-shape configuration.

Conduit symmetry indicators 650 may be attached to the vascular conduit by, for example, suturing, adhesive, or a combination thereof. In one embodiment, conduit symmetry indicators 650 are attached to the inner wall of the vascular conduit 602. In another embodiment, conduit symmetry indicators 650 are attached to the outer wall of the vascular conduit 602. In other embodiments, conduit symmetry indicators 650 are woven into the material of vascular conduit 602.

FIG. 6A illustrates vascular conduit 602 with conduit symmetry indicator device 650 in a symmetrical non-misshapen state, as it would appear prior to implantation. FIGS. 6B and 6C illustrate examples of the use of a conduit symmetry indicator device 650 to determine a misshapen conduit. FIGS. 6B and 6C illustrate vascular conduits 602B and 602C in an asymmetrical state. In FIG. 6B, misshapen conduit 602B causes conduit symmetry indicator devices 650B to become misshapen. As illustrated, during visualization of vascular conduit 602B, conduit symmetry indicator devices 650B appear as a slanted “T” thereby indicating to the practitioner that the conduit is not symmetrical. In FIG. 6C, misshapen conduit 602C causes conduit symmetry indicator devices 650C to become misshapen. As illustrated, during visualization of vascular conduit 602C, conduit symmetry indicator device 650C appears as a “T” having an arched portion thereby indicating to the practitioner that at least a portion of the conduit is not symmetrical.

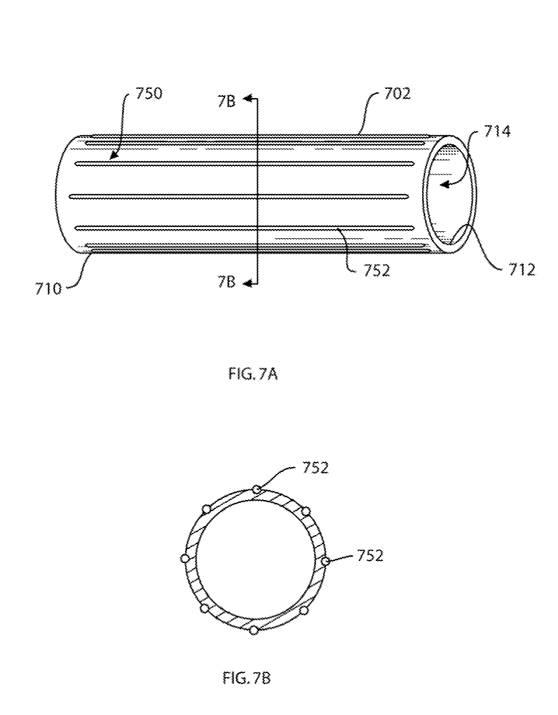

FIGS. 7A and 7B illustrate another embodiment of a vascular conduit 702 having a plurality of conduit symmetry indicator devices 750. Vascular conduit 702 comprises an elongate tubular member having an outer surface 710 and an inner surface 712, the inner surface defining a conduit lumen 714. In one embodiment, conduit 702 is the same as or similar to conduit 202, described above. FIG. 7B is a cross section of vascular conduit 702 taken along line 7B-7B illustrated in FIG. 7A.

Conduit symmetry indicator device 750 comprises a plurality of elongate members 752 attached to or embedded within the wall of vascular conduit 702. Elongate members 752 comprise metallic or polymeric radiopaque material having a high X-ray attenuation coefficient. Examples of suitable materials include, but are not limited to, barium sulfate and bismuth sub-carbonate for plastics, and gold and platinum for metals. Elongate members 752 comprise a filament of radiopaque material. The filament may be a wire or a plurality of wires braided into a filament. In another embodiment, elongate members 752 comprise a plurality of rigid radiopaque members disposed within the wall of vascular conduit 702.

Those with skill in the art will appreciate that the number and arrangement of the conduit symmetry indicator devices may vary depending on a particular application. It is contemplated that any arrangement of conduit symmetry indicator devices that provide a practitioner the ability to determine by visualization whether or not a conduit is misshapen is contemplated by the present invention.

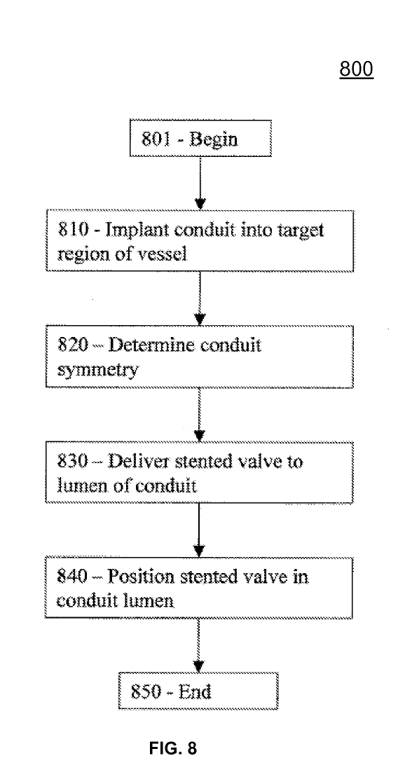

FIG. 8 is a flowchart illustrating method 800 for treating right ventricular outflow tract abnormalities by replacing a pulmonary valve, in accordance with the present invention. Method 800 begins at step 801. At step 810 a bioprosthetic conduit having at least one conduit symmetry indicator device is implanted into a target region of a vessel.

At step 820, conduit symmetry is determined. Conduit symmetry is determined by visualization of the at least one conduit symmetry indicator device. The conduit symmetry indicator device may be visualized using fluoroscopy, echocardiography, ultrasound, or other suitable means of visualization.

Next, a stented valve is delivered into a target site within a lumen of the bioprosthetic conduit, at step 830. In one embodiment, the stented valve is delivered percutaneously via a delivery catheter as are known in the art. In one embodiment, the target site within the conduit lumen comprises that portion of the lumen containing a pulmonary valve.

Optionally, prior to delivery of the stented valve to the target site at step 830, a symmetry corrective device is delivered to the target site. The corrective device is implanted to provide a symmetrical lumen prior to implantation of the stented valve. In one embodiment, symmetry corrective device is an expandable support structure. Corrective device may be balloon expandable or self-expanding. In one embodiment, the corrective device comprises a self-expanding framework composed of a biocompatible metal.

At step 840, the stented valve is expanded to position the stented valve within the conduit lumen. In one embodiment, the stented valve is expanded into position using a balloon. In another embodiment, the stented valve comprises a self-expanding stent that expands radially when released from the delivery catheter. In one embodiment, the stented valve expands radially when released from a restraining sheath of the delivery catheter. In those embodiments where a symmetry corrective device is used, the stented valve is expanded into contact with the corrective device. Method 800 ends at 850.

While the invention has been described with reference to particular embodiments, it will be understood by one skilled in the art that variations and modifications may be made in form and detail without departing from the spirit and scope of the invention.

1. A vascular valve replacement system, the system comprising:
a conduit comprising an elongate tubular member having an outer surface and an inner surface, the inner surface defining a conduit lumen;
at least one symmetry indicator attached to the elongate tubular member; and
a replacement valve device, the replacement valve device including a prosthetic valve connected to an expandable support structure, the replacement valve device positioned within the conduit lumen adjacent the inner surface.

2. The system of claim 1 wherein the at least one symmetry indicator comprises a framework having a plurality of spaced apart rings and a plurality of spaced apart elongate members attached to the plurality of rings.

3. The system of claim 2 wherein the plurality of rings and the plurality of elongate members comprise radiopaque filaments.

4. The system of claim 3 wherein the conduit comprises a woven material and the radiopaque filaments are interwoven into the woven material of the conduit.

5. The system of claim 3 wherein the conduit comprises a bioprosthesis and the radiopaque filaments are threaded through a conduit wall.

6. The system of claim 1 wherein the at least one symmetry indicator comprises a T-shaped radiopaque member attached to or imbedded within a wall of the conduit.

7. The system of claim 6 wherein the T-shaped radiopaque member comprises a plurality of filaments in a T-shaped configuration attached to the outer surface of the conduit.

8. The system of claim 1 wherein the T-shaped radiopaque member comprises a plurality of filaments in a T-shaped configuration attached to the inner surface of the conduit.

9. The system of claim 1 wherein the at least one symmetry indicator comprises a plurality of elongate members spaced apart around the circumference of the conduit, the plurality of elongate members parallel to a central axis of the conduit lumen.

10. The system of claim 1 further comprising a corrective device positioned within the conduit lumen between the inner surface of the conduit and an outer surface of the replacement valve device.

11. A prosthetic conduit device for treating a vascular condition, comprising:
a conduit comprising an elongate tubular member having an outer surface and an inner surface, the inner surface defining a conduit lumen; and
at least one symmetry indicator attached to the elongate tubular member.

12. The device of claim 11 wherein the at least one symmetry indicator comprises a framework having a plurality of spaced apart rings and a plurality of spaced apart elongate members attached to the plurality of rings.

13. The device of claim 12 wherein the plurality of rings and the plurality of elongate members comprise radiopaque filaments.

14. The device of claim 13 wherein the conduit comprises a woven material and the radiopaque filaments are interwoven into the woven material of the conduit.

15. The device of claim 13 wherein the conduit comprises a bioprosthesis and the radiopaque filaments are threaded through a conduit wall.

16. The device of claim 11 wherein the at least one symmetry indicator comprises a T-shaped radiopaque member attached to or imbedded within a wall of the conduit.

17. The device of claim 16 wherein the T-shaped radiopaque member comprises a plurality of filaments in a T-shaped configuration attached to the outer surface of the conduit.

18. The device of claim 11 wherein the at least one symmetry indicator comprises a plurality of elongate members spaced apart around the circumference of the conduit, the plurality of elongate members parallel to a central axis of the conduit lumen.

19. A method for treating a vascular condition, the method comprising:
inserting a conduit having a radiopaque conduit symmetry device into a target region of a vascular system, the conduit having an inner wall defining a conduit lumen;
visualizing the radiopaque conduit symmetry device;
determining conduit symmetry based on the visualization of the radiopaque conduit symmetry device;
delivering a stented valve into the conduit lumen, the stented valve including a prosthetic valve connected to an expandable support structure; and
expanding the stented valve into contact with the inner wall of the conduit.
